(19) United States
(12) Patent Application Publication
(10) Pub. No.: US 2008/0299636 A1
(43) Pub. Date: Dec. 4, 2008

(54) SYSTEM AND METHOD FOR INACTIVATING MICROORGANISMS WITH A FEMTOSECOND LASER

(76) Inventors: Kong-Thon Tsen, Tempe, AZ (US); Shaw-Wei D. Tsen, Baltimore, MD (US); Juliann G. Kiang, Potomac, MD (US)

Correspondence Address: SCHMEISER OLSEN & WATTS, 18 E UNIVERSITY DRIVE, SUITE # 101, MESA, AZ, 85201, US

(21) Appl. No.: 12/131,710

(22) Filed: Jun. 2, 2008

Related U.S. Application Data
(60) Provisional application No. 60/932,668, filed on Jun. 1, 2007.

(51) Int. Cl. C12N 13/00; H01S 3/10

(52) U.S. Cl. 435/173.1; 372/22

(57) ABSTRACT

The invention relates to a method of inactivating or diminishing the activity of microorganisms and an apparatus implementing that method. Microorganism activity is diminished through exciting the vibrational state of a microorganism with pulses of radiation femtoseconds in width. The wavelength of the pulses is in a range of the electromagnetic spectrum to which water is substantially transparent such as visible light. The pulses cause the microorganisms to vibrate such that their activities become diminished. A laser produces the pulses. A harmonic generator then acts upon the pulses to produce a scattering effect that is used to irradiate the microorganisms. One such laser is a titanium sapphire laser. One example of a harmonic generator is a nonlinear crystal such as a BBO crystal. The apparatus may also have a focusing lens such as a microscope objective that focuses the beam on a microorganism. The invention is effective for the inactivation of viruses or bacteria. The present invention may be utilized in order for extracting nucleic acid from microorganisms. The invention may be used in the manufacturing of vaccines. The invention enables the selective inactivation of target viruses and bacteria without causing cytotoxicity in mammalian cells.

### STATEMENT REGARDING FEDERALLY SPONSORED RESEARCH

The United States government has a paid-up license in this invention and the right in limited circumstances to require the patent owner to license to others on reasonable terms as provided by the terms of Grant No. DMR-0305147 awarded by the National Science Foundation and Grant No. RAB2CF awarded by the Armed Forces Radiobiology Research Institute, Uniformed Services University of the Health Sciences of the United States Department of Defense.

### CROSS REFERENCE TO RELATED APPLICATIONS

This application is a claims priority to U.S. Provisional Patent Application entitled “System and Method for diminishing the activity of microorganisms with a visible femtosecond laser,” Ser. No. 60/932,668, filed Jun. 1, 2007, the disclosure of which is hereby incorporated entirely herein by reference.

### BACKGROUND OF THE INVENTION

The invention pertains to the field of inactivating or diminishing the activity of microorganisms and more specifically, the use of impulsive stimulated Raman scattering with a visible or near-infrared femtosecond laser to bring about this inactivation or diminution.

Modern methods for inactivating or altering the activity of viruses and other microorganisms are not fully effective and additionally evoke problems of resistance and clinical side effects. Microorganisms may undergo genetic mutations that cause them to become resistant to antibiotics, antimicrobials, antiviral medications, cleaning products, the human immune system, ultraviolet (UV) treatment, irradiation, and other agents meant to inactivate them. In addition, the side effects of currently employed antimicrobial agents limit their use in a clinical setting.

One method that avoids the problems of resistance is microwave/ultrasonic absorption. This technique aims to excite vibrational modes in the microorganisms to such high amplitude as to lead to their inactivation/disintegration. Because mutation of microorganisms has no effect upon the vibrational frequency of their structural proteins, lipids, and carbohydrates including capsids, coat proteins, cell walls, cell membranes, and membrane incorporated proteins, the microorganisms cannot evade destruction through mutation. As a result, this method avoids generation or evolution of resistant strains of microorganisms and is effective for inactivating both wild type and mutated microorganisms.

A major setback for excitation through microwave/ultrasonic absorption is that water also absorbs in these vibrational frequencies which are typically in the range of 30 GHz to 300 GHz (See References 1 and 2 below.) Because water is often present where there are active microorganisms, much of the microwave/ultrasonic excitation energy that would induce vibrational energy of microorganisms is instead absorbed by the water. Such methods are thus not feasible for most applications. More efficient methods for inducing vibrations in microorganisms are thus required to fully implement this property.

Previous methods that used lasers to diminish the activity of microorganisms, employed a UV laser which damaged the protein structure of the microorganisms. Other methods involved lasers utilizing different wavelengths. However, these techniques require a great deal of power and damage desirable materials such as human cells, platelets, and proteins in addition to killing the microorganisms.

So as to reduce the complexity and length of the Detailed Specification, and to fully establish the state of the art in certain areas of technology, Applicants herein expressly incorporate(s) by reference all of the following materials identified in each numbered paragraph below.
1. Ford L H, Estimate of the vibrational frequencies of spherical virus particles, Phys. Rev. E67, 051924-1-051924-3 (2003).
2. Tsen K T, Dykeman E C, Sankey O F, Lin N-T, Tsen S-W D, Kiang J G: Observation of the low frequency vibrational modes of bacteriophage M13 in water by Raman spectroscopy. Virology J, 3:79-1-79-11 (2006).
3. Cerf R. et al, Ultrasonic Absorption Evidence of Structural Fluctuations in Viral Capsids, PNAS (USA), 76 1780-1782 (1979).
4. Michaels B et al, Ultrasonic absorption in tobacco mosaic virus and its protein aggregates, J Mol Biol 181 103-110 (1985).
5. Yan Y X et al, Impulsive stimulated scattering: General importance in femtosecond laser pulse interactions with matter, and spectroscopic applications, J Chem Phys, 83 5391-5399 (1985).
6. Tsen K T, Ultrafast dynamics in wide bandgap wurtzite GaN, Ultrafast Physical Processes in semiconductors, K T Tsen ed., 67 109-149 Semiconductors and Semimetals, Academic Press, New York, (2001).
7. Tsen K T, Electron velocity overshoot, ballistic electron transport and non-equilibrium phonon dynamics in nanostructure semiconductors, Ultrafast Physical Processes in Semiconductors, K T Tsen ed., 67 191-259, Springer-Verlag, New York (2001).
8. Tsen K T, Optical studies of electric field induced electron and hole transient transports and optical phonon instability in semiconductor nanostructures. Ultrafast Dynamical Processes in Semiconductors, K T Tsen ed., 92 193-258, Topics in Applied Physics, Spinger-Verlag, Heidelberg (2004).
9. Tsen K T, Optical studies of carrier dynamics and non-equilibrium optical phonons in nitride based semiconductors, Non-equilibrium Dynamics of Semiconductors and Nanostructures, 180-213, K T Tsen ed. CRC Press, New York (2005).
10. Nelson K A et al, Optical generation of tunable ultrasonic waves, J Appl Phys 53 1144-1149 (1982).
11. Miller R J D et al, Laser-induced ultrasonics: A dynamic holographic approach to the measurement of weak absorptions, optoelastic constants acoustic attenuation, Chem Phys 72 371-379 (1982).
12. Nelson K A, Stimulated Billouin scattering and optical excitation of coherent shear waves, J Appl Phys 53 6060-6063 (1982).
13. Tsen K T et al, Raman scattering studies of the low-frequency vibrational modes of bacteriophage M13 in water—observation of an axial torsion mode, Nanotechnology 17, 5474-5479 (2006).
14. Robinson M M et al, Picosecond impulsive stimulated brillouin scattering: Optical excitation of coherent transverse acoustic waves and application to time-domain investigations of structural phase transitions. Chem Phys Lett 112 491-496 (1984)
15. De Silvestri S et al, Femtosecond time-resolved measurements of optic phonon dephasing by impulsive stimulated raman scattering in an α-perylene crystal from 20 to 300K, Chem Phys Lett 116 146-152 (1985).
16. Tsen K T et al, Observation of the low frequency vibrational modes of bacteriophage M13 in water by Raman spectoscropy, Virology J 3 79-1-79-11 (2006).
17. Tsen K T et al, Probing the low-frequency vibrational modes of viruses with Raman scattering—bacteriophage M13 in water, J Biomedical Optics 12 024009-1-024009-6 (2007).
18. Tsen K T et al, Inactivation of viruses by coherent excitations with a low power visible femtosecond laser, Virology J. 4, (50)1-5 (2007).
19. Liu J L et al, Intersubband absorption in boron-doped multiple Ge quantum dot, Appl Phys Lett, 74 185-187 (1999).
20. Liu J L et al, Observation of intersub-level transitions in modulation doped Ge quantum dots, Appl Phys Lett, 75 1745-1747 (1999)
21. Kamins T I et al, Lithographic positioning of self assembled Ge islands on Si, Appl Phys Lett 71 1201-1203 (1997)
22. Balandin A et al, Issues of practical realization of a quantum dot register for quantum computing, J Electronics Materials 29 549-553 (2000).
23. Shenton W et al, Inorganic-organic nanotube composites from template mineralization of tobacco mosaic virus, Adv Mater 11 253-256 (1999)
24. Flynn C E et al, Viruses as vehicles for growth, organization and assembly of materials, Acta Materialia 51 5867-5880 (2003).
25. Mao C et al, Virus-based toolkit for the directed synthesis of magnetic and semiconducting nanowires, Science 303 213-217 (2004).
26. Knez M et al, Spatially selective nucleation of metal clusters on the tobacco mosaic virus, Adv Funct Mater 14 116-124 (2004).
27. Tuma R et al, Raman Spectroscopy of Viruses, in Handbook of Vibrational Spectroscopy, Chalmers J M et al eds. John Wiley & Sons, Chichester (2002).
28. Eisenstark A, Bacteriophage techniques, in Methods in Virology Volume I 449-524, Maramorosch et al eds. Academic Press, New York (1967).
29. Tsen K T et al, Electron-optical phonon interactions in ultrathin GaAs—AlAs multiple quantum well structures, Phys Rev Lett 67 2557-2560 (1991)
30. Marvin D A et al, Molecular structure and fd (f1 M13) filamentous bacteriophage refined with respect to X-ray fibre diffraction and solid-state NMR data supports specific models of phage assembly at the bacterial membrane, J Mol Biol 355 294-309 (2006).
31. Balandin A et al, Vibrational modes of Nano-Template Viruses, Journal of Biomedical Nanotechnology 1 90-95 (2005).
32. Graff K F, Wave Motion in Elastic Solids, Ohio State University Press, New York (1991).
33. Landau L D et al, Theory of Elasticity, Third Edition, Pergamon Press London (1986).
34. Tachibana M et al, Sound velocity and dynamic elastic constants of lysozyme single crystals, Chem Phys Lett 332 259-264 (2000).
35. Yu P Y et al, Fundamentals of Semiconductors—Physics and Materials Properties 2nd Edition, 362-371, Springer-Verlag, Berlin (1999).
36. Go S et al, Bond Charge, Bond Polarizibility and Phonon Spectra in Semiconductors, Phys Rev Lett 34 580-583 (1975).
37. Moss B, Vaccinia virus, a tool for research and vaccine development, Science 1662-1667 (1991).
38. Bournsell M E G et al, Construction and characterization of a recombinant vaccinia virus expressing human papillomavirus proteins for immunotherapy of cervical cancer, Vaccine 14 1485-1494 (1996).
39. Messing J et al, Filamentous coliphage M13 as a cloning vehicle: insertion of a HindII fragment of the lac regulatory region in M13 replicative form in vitro, PNAS USA 74 3642-3646 (1977).
40. Merril C R et al, Long circulating bacteriophage as antibacterial agents, Microbiology 93 3188-3192 (1996).
41. Knez M et al, Biotemplate of 3-nm nickel and cobalt nanowires, Nano Lett 3 1079-1082 (2003).
42. Liu W L et al, Assembly and characterization of hybrid virus-inorganic nanotubes, Appl Phys Lett 86 253108 (2005)
43. Fonoberov et al, Low-frequency vibrational modes of viruses used for nanoelectronic self-assembly, Phys Status Solidi B Rapid Res Notes 12 R67-R69 (2004).
44. Fonoberov et al, Phonon confinement effects in hybrid virus-inorganic nanotubes for nanoelectronic applications, Nano Lett 5 1920-1923 (2005).
45. Snoke et al, A bond polarizibility model for the C₆₀ Raman spectrum, Solid State Commun, 87 121-126 (1993).
46. Dong J et al, Chemical trends of the rattling phonon modes in alloyed germanium cathrates, J Appl Phys 87 7726-7734 (2000).
47. Guha et al, Empirical bond polarizibility model for fullerenes, Phys Rev B 53 13106-13114 (1996).
48. Shen Y R, The Principles of Nonlinear Optics, Wiley, New York (1984).
49. Austen D H, et al eds., Ultrafast Phenomena IV, Springer, Berlin (1984).
50. Shen Y R, Prog. Quantum Electron 4 207 (1976).
51. Auston D H et al, Phys Rev Lett 53 1555 (1984).
52. Shen Y R et al, Phys Rev 163 224 (1967).
53. Laubereau A et al, Rev. Mod Phys 50 607 (1978).
54. Shen Y R et al, Phys Rev A 137 1787 (1965).
55. Kosic T J et al, Chem Phys Lett 96 57 (1983).
56. Cameron A et al, Acta Crystallogr 18 636 (1965).
57. Kaiser W et al, in Laser Handbook Vol 2, Arrechi F T et al eds., North-Holland, Amsterdam (1972).
58. Dissado L A et al, Chem Phys 86 375 (1984).
59. Dissado L A et al, Chem Phys Lett 87 74 (1982).
60. Collins M A et al, Chem Phys 54 305 (1981).
61. Grover M et al, J Chem Phys 54 4843 (1971).
62. Gehrig G A et al, Rep Prog Phys 38 1 (1975).
63. Tanaka J et al, Bull Chem Soc Jpn 36 1237 (1963).
64. Cohen M D et al, Chem Phys 27 211 (1978).
65. Nelson K A et al, Chem Phys Lett 64 88 (1979).
66. Walker B et al, Chem Phys 97 177 (1985).
67. Prasad P N et al, Mol Cryst Liq Cryst 93 25 (1983).
68. Sukenik J A et al, J Am Chem Soc 99 851 (1977).
69. Dwarakanath et al, J Am Chem Soc 102 4254 (1980).
70. Merski J et al, J Chem Phys 75 3691, 3705, 3719, 3731 (1981).
71. Luty T et al, J Chem Phys 81 520 (1984).
72. Luty T et al, J Chem Phys 82 1515 (1985).
73. Nelson K A et al, J Chem Phys 72 5202 (1980).
74. Shriver D F et al, in Advances in IR and Raman Spectroscopy, Clark R J H ed., 6 127 Heydon and Son, London (1980).
75. Corn R M et al, J Chem Phys 12 5231 (1984).
76. Sidorov N V et al, Mol Cryst Liq Cryst 90 185 (1983).
77. Baughman R H et al, in Synthesis and Properties of Low-dimensional Materials, Miller J S et al ed., 75 Academy of Science Press, New York (1978).
78. Gross H et al, Chem Phys Lett 95 584 (1983).
79. Warshel A et al, Chem Phys 6 463 (1974).
80. Cotton et al, Advanced Inorganic Chemistry 4th ed., Interscience, New York (1980).
81. Wrighton M S, in Reactivity of Metal-Metal Bonds, Chisholm M H ed., ACS Symp Ser 155 85, American Chemical Society, Washington D.C. (1981).

Applicants believe that the material incorporated above is “non-essential” in accordance with 37 CFR 1.57, because it is referred to for purposes of indicating the background of the invention or illustrating the state of the art. However, if the Examiner believes that any of the above-incorporated material constitutes “essential material” within the meaning of 37 CFR 1.57(c)(1)-(3), applicants will amend the specification to expressly recite the essential material that is incorporated by reference as allowed by the applicable rules.

### BRIEF SUMMARY OF THE INVENTION

The present invention provides among other things an apparatus and method for inactivating or diminishing the activity of microorganisms in a manner that minimizes side effects and that is unaffected by the development of resistance in response to selective pressure. “Inactivating or diminishing the activity of microorganisms” refers to “inhibiting/reducing the growth, survival, function, and/or infectivity of microorganisms”.

It is an object of the invention to manipulate, control, and inactivate microorganisms.

It is an object of the invention to excite vibrational states in microorganisms in the presence of water.

It is an object of the invention to excite vibrational states in microorganisms using low energy electromagnetic waves in the IR and visible range.

It is an object of the invention to produce large-amplitude vibrational modes on structures essential for the functionality of microorganisms using Impulsive Stimulated Raman Scattering (ISRS).

It is an object of the invention to excite coherent acoustic Raman-active vibrational modes on structures essential for the functionality of microorganisms through ISRS to a state that leads to a selective diminution of their activity that includes their inactivation through forced mechanical acoustic vibrations.

It is an object of the invention to inactivate viruses and/or bacteria such that the component parts of the virus and/or bacteria become altered or disassociated in such a way that they may be used for vaccination.

It is an object of the invention to selectively inactivate microorganisms with no effects upon eukaryotic cells.

It is an object of the invention to inactivate bloodborne pathogens in the blood while sparing key blood cells and blood components.

The above and other objects will be achieved using devices involving a laser that is able to generate pulses of radiation femtoseconds in width with wavelengths that correspond to regions of the electromagnetic spectrum to which water is substantially transparent. The device also uses a harmonic generator such as a nonlinear BBO crystal that produces a scattering effect that then irradiates the microorganism by exciting the microorganism's vibrational state. The device may also have a focusing lens such as a microscope objective. The laser may be a titanium sapphire laser. Targeted microorganisms include viruses and bacteria and protozoa.

The above and other objects will be achieved using methods involving exciting the vibrational state of the microorganism, with femtosecond pulses of radiation in a range of the electromagnetic spectrum to which water is substantially transparent which in turn produces mechanical acoustic excitations in the microorganism. The mechanical acoustic excitations then lead to diminution of the activity of the microorganism when the beam is focused upon the microorganism. The activity of the microorganism may be diminished to such a degree by the mechanical acoustic excitiations that the microorganism becomes inactivated.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the “special” definition of that term and explain how it differs from the plain and ordinary meaning. Absent such clear statements of intent to apply a “special” definition, it is the inventors' intent and desire that the simple, plain and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. §112, ¶ 6. Thus, the use of the words “function,” “means” or “step” in the Detailed Description or Description of the Drawings or claims is not intended to somehow indicate a desire to invoke the special provisions of 35 U.S.C. §112, ¶ 6, to define the invention. To the contrary, if the provisions of 35 U.S.C. §112, ¶ 6 are sought to be invoked to define the inventions, the claims will specifically and expressly state the exact phrases “means for” or “step for, and will also recite the word “function” (i.e., will state “means for performing the function of [insert function]”), without also reciting in such phrases any structure, material or act in support of the function. Thus, even when the claims recite a “means for performing the function of . . . ” or “step for performing the function of . . . ,” if the claims also recite any structure, material or acts in support of that means or step, or that perform the recited function, then it is the clear intention of the inventors not to invoke the provisions of 35 U.S.C. §112, ¶ 6. Moreover, even if the provisions of 35 U.S.C. §112, ¶ 6 are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

### BRIEF DESCRIPTION OF THE SEVERAL VIEWS OF THE DRAWINGS

A more complete understanding of the present invention may be derived by referring to the detailed description when considered in connection with the following illustrative figures. In the figures, like reference numbers refer to like elements or acts throughout the figures.

FIG. 1 depicts a form of the invention that may be used to diminish the activity of M13 bacteriophage.

FIG. 2A depicts the results of plaque forming assays on M13 bacteriophage at a titer of 1×10³ pfu without laser irradiation

FIG. 2B depicts the results of plaque forming assays on M13 bacteriophage at a titer of 1×10³ pfu with laser irradiation

FIG. 3A depicts the results of plaque forming assays on M13 bacteriophage at a titer of 5×10² pfu without laser irradiation

FIG. 3B depicts the results of plaque forming assays on M13 bacteriophage at a titer of 5×10² pfu with laser irradiation.

FIG. 4 shows the results of plaque forming assays on M13 bacteriophage at a titer of 1×10³ pfu with varying excitation laser power densities.

FIG. 5 is a table showing the results of different pulse widths upon inactivation of microorganisms.

Elements and acts in the figures are illustrated for simplicity and have not necessarily been rendered according to any particular sequence or embodiment.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention. It should be noted that there are many different and alternative configurations, devices and technologies to which the disclosed inventions may be applied. The full scope of the inventions is not limited to the examples that are described below.

The invention excites the vibrational states of microorganisms using ultrashort, low energy pulses in ranges of the electromagnetic spectrum to which water is essentially transparent. One such range is the spectrum of visible light. Impulsive Stimulated Raman Scattering (ISRS) may be used to produce large-amplitude vibrational modes in molecules present liquid solution as well as lattice vibrations in solid state systems (See reference 3).

ISRS has been successfully demonstrated in molecular as well as solid state systems (see references 8-12). In molecular systems, ISRS with a single laser-beam excitation is a forward-scattering process which is stimulated because the Stokes frequency is contained within the spectral width of the excitation pulse. Furthermore, ISRS is a process through which excitation of coherent lattice or molecular vibrations would take place whenever a sufficiently short laser pulse passes through a Raman-active solid or molecular liquid or gas. For a single beam excitation, if the dampening is ignored, then the amplitude (R₀) of the displacement away from the equilibrium intermolecular distance caused by ISRS can be shown to be given by:

R₀=4π I(δα/δR)₀e^{-ω}^{₀}^{²}^{τ}^{_{L}}^{²}^{/4}/mω₀nc (1);

where I is the power density of the excitation laser; α is the polarizability of the medium; R is the displacement away from the equilibrium intermolecular distance; δα/δR is proportional to the Raman scattering cross section; ω₀ is the angular frequency of the coherent vibrational excitation; τ_{L} is the full width at half maximum (FWHM) of the pulse width of the excitation laser; m is the molecular mass; n is the index of refraction; and c is the speed of light (See reference 3).

Based upon the above equation, larger Raman cross sections, higher laser power densities, as well as lower vibrational frequencies, would contribute to greater excited vibrational amplitude. For many Raman scattering cross sections with a sufficiently low vibrational frequency and a reasonable excitation power density, an amplitude of vibrational displacement in the 0.01 to 1 Å range could be achieved through ISRS. The derivative of the polarizibility of the excited molecular system with respect to the molecular displacement should be larger than 10⁻¹⁶ cm².

Amplitudes of vibrational displacement in this range may be used to create vibrations in microorganisms. For example, M13 bacteriophages display low frequency (≅8.5 cm⁻¹) vibrational mode associated with the axial torsion vibrations of the viral capsid. (See references 13 and 14). With ultrashort pulse laser excitation, the amplitude of this vibrational mode may be coherently excited by ISRS to the extent that the activity of the microorganisms is diminished.

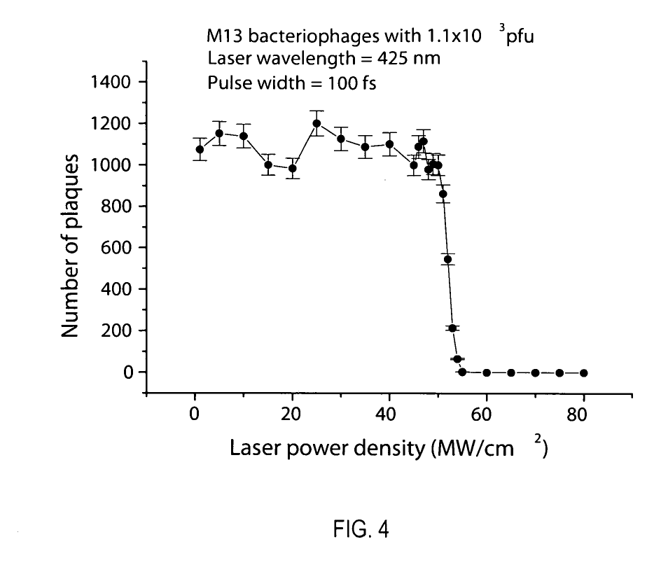

A threshold amplitude of the vibrational mode must be exceeded for the activity to be diminished. For example, referring to FIG. 4, the activity of M13 bacteriophages is not affected until an excitation laser power density of about 50 MW/cm² is exceeded. Additionally, even if the threshold for excitation laser power density is exceeded, if the pulse width is longer than 800 femtoseconds, then activity is not affected. Other microorganisms may have their activities diminished at different amplitudes from M13 bacteriophage with such amplitudes approximated by the above equation.

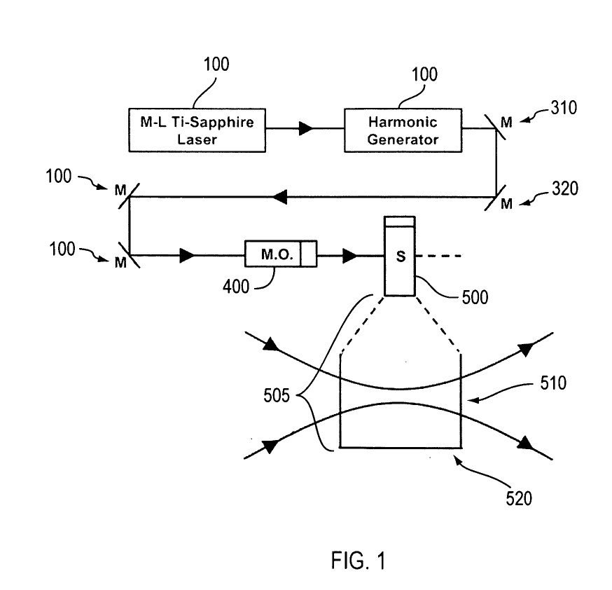

In one form of the invention, a visible femtosecond laser system excites a coherent acoustic Raman-active vibrational mode in microorganisms through ISRS to a state that leads to a selective diminution of their activity that includes their inactivation through mechanical acoustic excitations. Referring now to FIG. 1, a laser 100 produces pulses on the order of femtoseconds with a given repetition rate (see references 4-7). A second harmonic generator 200 is used to irradiate the sample. Mirrors 310, 320, 330 and 340 reflect the beam to a focusing lens 400 that focuses the beam into the sample container 500. In a close-up view of the sample container 505, the focus of the beam defines the sample container into an area in which the beam is intensely focused 510 and an area in which the beam is less intensely focused 520.

One example of a laser that can be used in the invention is a diode-pumped cw mode-locked Titanium-sapphire laser. However, other femtosecond lasers may be employed. Such femtosecond lasers include ring lasers, argon-pumped dual-jet dye lasers, or the second harmonic output of a YAG lasers, ultrashort pulsed fiber lasers. Other Ti:sapphire lasers include integrated pump lasers such as the Pallas-LP from Time-Bandwidth Products, cavity-dumped femtosecond Ti:sapphire laser systems such as the Tiger-CD tunable Nd: glass lasers such as the GLX-200 from Time-Bandwidth Products, or passively mode-locked thin disk lasers such as the Fortis from Time-Bandwidth Products.

The laser is set to produce a continuous train of pulses at a set repetition rate. Preferably, the pulses are about 80 femtoseconds in width and the repetition rate is about 80 MHz, the wavelength is about 425 nm, and the power is about 40 mW. However, other settings may be used. For example, pulse widths from about one attosecond to about one picosecond may be used and wavelengths from about 400 nm to about 900 nm may be used.

Preferably, the harmonic generation system may be a BBO nonlinear crystal, but other nonlinear crystals for doubling the near infrared to visible light may be used. These alternatives include LBO, LiNbO3, KTP, LiTaO3, KNbO3, KDP, CLBO, BIBO, CBO, ZGP, AgGaS2, AgGaSe2, CdSe, and GaAs nonlinear crystals.

The focusing lens can be a microscope objective with an extra long working distance, preferably about 2.0 cm. However, other focusing lenses with different working distances may be employed. Focusing lenses may include compound lenses or fiber optics lenses.

The invention may be adjusted so as to focus the laser upon a structure that has microorganisms on its surface, to disrupt the structure of the microorganism in a characteristic way, or to diminish the activity of microorganisms present in a liquid, solid, or gas.

For a more detailed discussion of the types of femtosecond lasers, their settings and properties, see the following texts, hereby incorporated by reference:
82. Claude Rulliere, Femtosecond Laser Pulses: Principles and Experiments (Advance Texts in Physics) 2nd Ed. (2005).
83. Jean Claude Diels and Wolfgang Rudolph, Ultrashort Laser Pulse Phenomena: Fundamentals, Techniques and Applications on a Femtosecond Time Scale (Optics and Photonics Series), Academic Press (1996).
84. Sigrid Avrillier, Femtosecond Laser Applications in Biology, Proceedings of SPIE, Volume 5463 (2004).

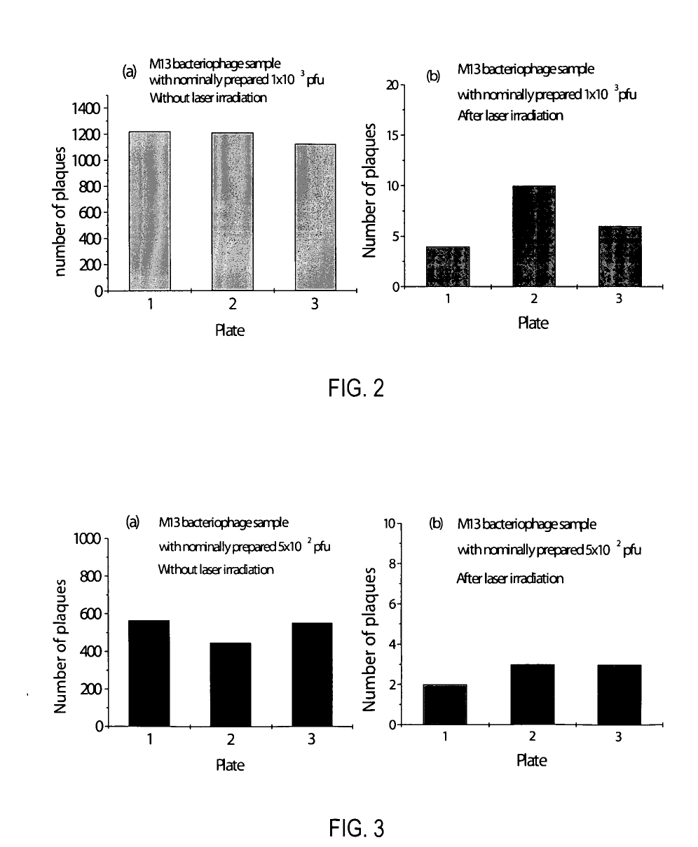

Referring now to FIGS. 2A and 2B, three separate plates prepared with 1×10³ pfu of M13 bacteriophage were irradiated with a 1 nj/pulse femtosecond laser with a wavelength of 425 nm and a pulse width of 100 femtoseconds. M13 phages were inactivated to statistical significance (note differences in scale for the y-axis of the graphs.)

Referring now to FIGS. 3A and 3B, three separate plates prepared with 5×10² plaque forming units M13 bacteriophage were irradiated with a 1 nj/pulse femtosecond laser with a wavelength of 425 nm and a pulse width of 100 femtoseconds. M13 phages were inactivated to statistical significance (note differences in scale for the y-axis of the graphs.)

Referring now to FIG. 4, M13 phage were plated at 1.1×10³ pfu per plate and subjected to pulses at a wavelength of 425 nm with a pulse width of 100 fs. Different laser power densities were used. At least for M13 phage, the laser was not effective at inactivating the phage until a laser power density of at least 45 MW/cm² was reached. These settings may not be the same for all microorganisms.

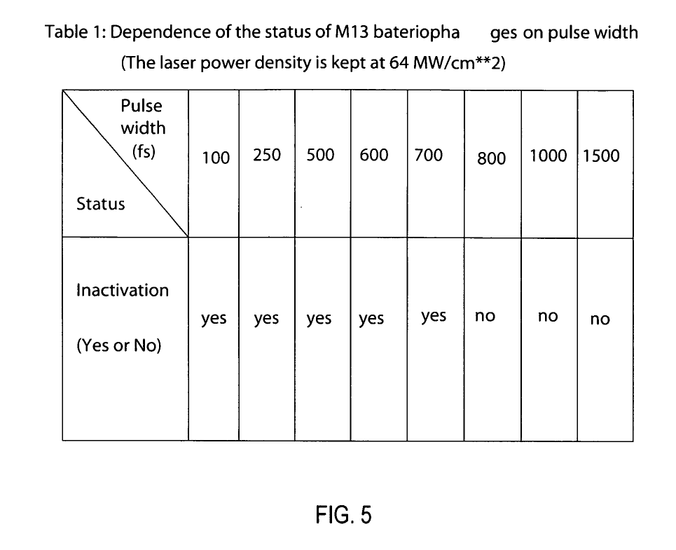

Referring now to FIG. 5, the effect of different pulse widths upon M13 phage irradiated with pulses of 425 nm and a power of 64 MW/cm² is determined. Notice that inactivation does not become effective unless the pulse length is less than 800 femtoseconds. These settings may not have the same effectiveness for all microorganisms.

Accordingly to particular embodiments of the present invention, by use of impulsive stimulated Raman scattering, the present invention may selectively inactivate viruses with a femtosecond laser. Samples of M13 bacteriophages were plated at 1.1×10⁷ pfu/ml and subjected to pulses at a wavelength of 425 nm with a pulse width of 100 fs. Different laser power densities were used. At least for M13 bacteriophage, the laser was not effective at inactivating the phage until a laser power density of at least 49 MW/cm² was reached. For a very low power visible femtosecond laser, viruses such as the M13 bacteriophages may be inactivated through ISRS process. These settings may not be the same for all microorganisms, however, it is understood that the present invention may be utilized to selectively inactivate microorganisms while leaving the sensitive materials unharmed by manipulating and controlling with femtosecond laser system.

Accordingly for different types of microorganisms, the wavelength and pulse width may be appropriately selected with a corresponding window in power density that enables the selective inactivation of target viruses and bacteria without causing cytotoxicity in mammalian cells.

For a more detailed discussion of selectively inactivating microorganimsas, see the following texts, hereby incorporated by reference:
85. K. T. Tsen et al, Inactivation of viruses with a femtosecond laser via impulsive stimulated Raman scattering, Proceedings of SPIE, Volume 6854, 68540N (2008).
86. K. T. Tsen et al, Selective inactivation of micro-organisms with near-infrared femtosecond, J. Phys.: Condensed Matter 19, 472201 (1-7) (2007).
87. K. T. Tsen et al, Inactivation of viruses by laser-driven coherent excitations Via impulsive stimulated Raman scattering process, Journal of Biomedical Optics 12, 064030 (1-7) (2007).
88. K. T. Tsen et al, Selective inactivation of human immunodeficiency virus with subpicosecond near-infrared laser pulkses, J. Phys.: Condensed Matter, 20, 252205 (1-4) (2008).

Other embodiments of the present invention may be utilized in order for extracting nucleic acid from microorganisms. Through excitation of the microorganisms the present invention allows for the formation of perforation on the protein coat of microorganisms, as a result, nucleic acid within the microorganisms can be released that can be collected and analyzed.

Additionally, because the amplitude of the vibrations varies continuously with the laser power density, other embodiments of the present invention may include excitation of microorganisms until the microorganism reaches a state where it is inactive, but remains intact in an altered or fractured state. It is contemplated that the microorganisms may then be used in the manufacturing of vaccines.

The embodiments and examples set forth herein were presented in order to best explain the present invention and its practical application and to thereby enable those of ordinary skill in the art to make and use the invention. However, those of ordinary skill in the art will recognize that the foregoing description and examples have been presented for the purposes of illustration and example only. The description as set forth is not intended to be exhaustive or to limit the invention to the precise form disclosed. Many modifications and variations are possible in light of the teachings above without departing from the spirit and scope of the forthcoming claims.

We claim:

1. An apparatus that diminishes the activity of a microorganism comprising:
a laser having an output configured to generate femtosecond pulses in a range of the electromagnetic spectrum to which water is substantially transparent; and
a harmonic generator operating on the femtosecond pulses to produce a scattering effect thus irradiating the microorganisms by exciting the microorganism's vibrational state.

2. The apparatus of claim 1 wherein the laser comprises a titanium sapphire laser or ultrashort pulsed fiber laser.

3. The apparatus of claim 1 wherein the femtosecond pulses have a power within the range of 1 nanojoule per pulse to 1 millijoule per pulse.

4. The apparatus of claim 1 wherein the harmonic generator comprises a nonlinear crystal.

5. The apparatus of claim 1 wherein the harmonic generator comprises a BBO nonlinear crystal.

6. The apparatus of claim 1 wherein the range of the electromagnetic spectrum to which water is substantially transparent comprises the range for infrared and visible lights.

7. The apparatus of claim 1 wherein the microorganism comprises a virus.

8. The apparatus of claim 1 wherein the microorganism comprises a bacterium.

9. The apparatus of claim 1 further comprising a focusing lens that focuses the scattered pulses upon the microorganism.

10. The apparatus of claim 9 wherein the focusing lens comprises a microscope objective.

11. A method for diminishing the activity of a microorganism comprising:
irradiating the microorganism into a vibrational state with femtosecond pulses of radiation in a wavelength that is in a range of the electromagnetic spectrum to which water is substantially transparent, thereby generating manual acoustic excitations in the microorganism that diminish the activity of the microorganism.

12. The method of claim 11 further comprising focusing the beam of the excitation source upon the microorganism.

13. The method of claim 11 wherein the microorganism is inactivated.

14. The apparatus of claim 1 wherein the microorganism comprises a protozoon.
